(19) United States
(12) Patent Application Publication
(10) Pub. No.: US 2008/0066770 A1
(43) Pub. Date: Mar. 20, 2008

(54) Methods for diagnosis and treatment of temporomandibular joint dysfunction and orofacial pain

(76) Inventor: Steven R. Olmos, Alpine, CA (US)

Correspondence Address: Mitch C. Wallis, 3657 Vermont Street, San Diego, CA, 92103, US

(21) Appl. No.: 11/523,815

(22) Filed: Sep. 19, 2006

(51) Int. Cl. A61B 19/00

(52) U.S. Cl. 128/898

(57) ABSTRACT

A method and system of diagnosis and treatment of temporomandibular joint dysfunction and orofacial pain, preferably without surgery, comprises preferably a non-invasive evaluation of neuroanatomy, tissue pathology, and tonus of muscles to prescribe highly specialized orthotic therapy, sometimes in combination with physical therapy and arthocentesis.

### DETAILED DESCRIPTION OF THE INVENTION

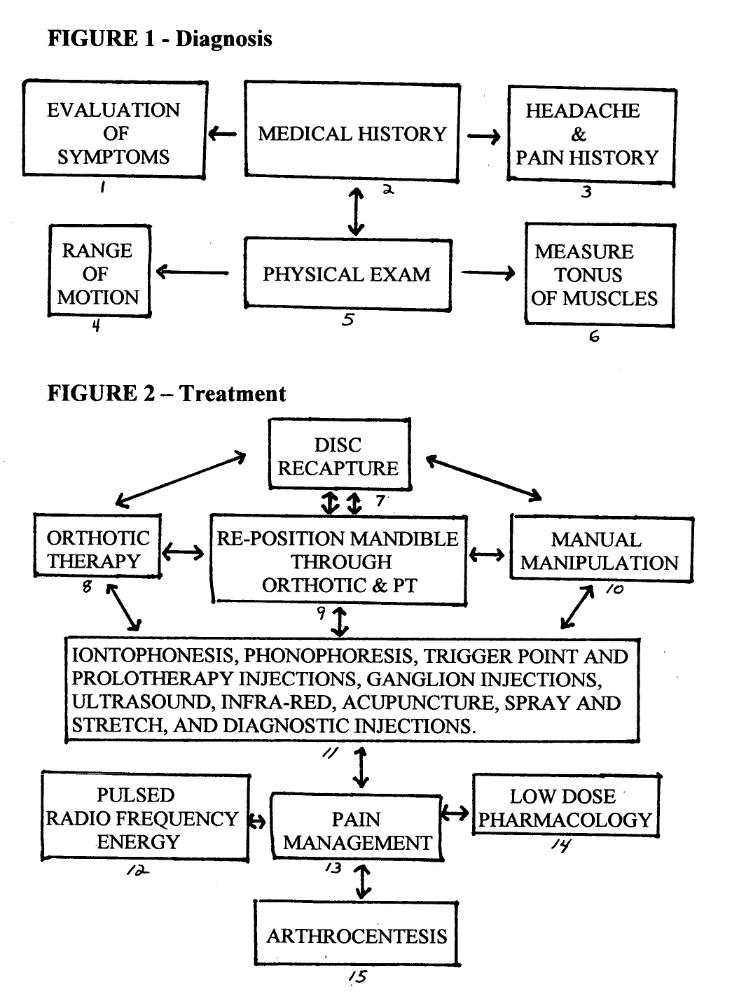

The first step in this method is to identify whether there are TM injuries and if so, whether they are primary (FIG. 1). TM joint symptoms may be secondary or tertiary, and in this case the patient should be referred to a specialist. It is vital to the success of this invention that only primary TM injuries are treated, otherwise treatment may exacerbate the problem.

The following methods must be employed to properly diagnose TMJ: obtaining a complete patient medical history 2 including a description of chief complaints 1, prior treatment, medication usage, past trauma including a complete headache and pain history 3 including location, severity, and frequency and a determination of whether symptoms are chronic or acute, diurnal or nocturnal; and conducting a comprehensive physical examination 5 to locate the source of pain and identify any dysfunction in the masticatory system including a general examination of the face, head, and neck, cranial nerve screening, autonomic nervous system evaluation, intra-oral evaluation, range of motion 4, Joint Vibration Analysis, posture evaluation, recording of vitals, and a complete series of radiographic imaging including the measuring of tonus of muscles 6 by using electromyography and the measuring of mandibular movement by using electrognathology, autonomic testing 7 used to locate the primary area of injury, and the phonetic ‘S’ position of speech 8 used to determine proper condyle fossa relationship and to improve the passage of airway.

In order to utilize these methods of diagnosing TMJ, the health practitioner must be familiar with a healthy, normal functioning temporomandibular joint to use as a reference point. Essential to employment of the present invention is a thorough study and understanding of the embryology, development, and function of this joint and relative anatomy, including the function and need of muscles both at the gross and molecular levels, neurology and its direction of muscle movement and tonus, somatic muscular neurology, autonomic innervation, and sensory mechanisms.

Neuroanatomy and an understanding of the component parts of the central nervous system form the underlying structure of this method of diagnosis. To utilize the present invention, the health practitioner must understand each of the component part's function and pathways of ascending and descending neurology. The understanding of the blood supply to the brain and its innervation is required to properly diagnose and make the connection between the trigeminal nervous system and the ‘Circle of Willis’ in migraines.

A thorough understanding of the autonomic nervous system (parasympathetic and sympathetic systems) is necessary for the prescription of medicines and to triage the injuries in order to prioritize treatment and determine whether therapeutic (rehabilitative) or symptomatic (pain relief) treatment is proper.

Non-invasive objective evaluation of tissue pathology is imperative for proper diagnosis. Joint Vibration Analysis is an important aid in the evaluation of tissues, both hard and soft, of the TM joint. Electrognatholoy is important in objectively measuring mandible movement for documentation or to establish a neuromuscular bite registration and for measuring the tonus of muscles.

All aspects of clinical examination are crucial to a proper diagnosis. Especially important is knowledge of cranial nerves. Posture is also an important diagnostic tool.

The brain of each patient prioritizes injuries. It is necessary for the health practitioner to understand this prioritization in order to make an effective diagnosis. If the health practitioner treats injuries in order of priority he can achieve a constant reduction of symptoms without causing damage to other structures or producing or magnifying symptoms in other location.

Once primary TM injury is identified, an evaluation of the level of degeneration of the autonomic nervous system must be made. These tests are used both to determine the priority of symptoms and to gauge whether the specific orthotic prescribed for the patient is effective and whether it continues to be effective.

The phonetic ‘S’ position of speech is used to locate the proper condyle fossa position relationship. Speech is a neutral neural and motor function and when a patient makes the ‘S’ sound their tongue and mandible are level. The condyle is downward and forward which allows for decompression with minimal elevator muscle tonus, thus being comfortable. In bringing the tongue forward it also allows for an improved airway in patients with compromised airways.

Treatment

The benefits of non-surgical treatment (FIG. 2), and its method of administration, depend to a large extent on the efficacy of the diagnosis. The treatment set forth below has been shown to produce substantial benefits of relieving both the symptoms and the origins of TMJ when used in combination with an accurate diagnosis. However, this treatment may be modified by future scientific study.

The present invention embodies, as a primary treatment method, the repositioning of the mandible 11 to re-establish proper capsular and dental relationships.

Discs are recaptured 9 using principles of gentle body mechanics.

The mandible is re-positioned through orthotic and physical therapy 11 to restore function and reduce symptoms. The primary method is performed by manual manipulation 12 consisting of depressing the mandible and simultaneously rotating the mandible to the right and the left while maintaining a downward force.

Specifically, placing the thumbs at the junction of the ramus and body of the mandible (intra-orally over the molars) and the index finger just superior to the angle of the mandible with the remaining fingers on the body of the mandible (extra-orally), downward pressure can be applied. The patient must be instructed to relax, as any manual manipulation will be countered by the elevator muscles.

Oral sedation, inhalation sedation, and IV sedation are useful in relaxing the elevator muscles and aid in the recapturing process. In addition to depressing the mandible, it is necessary to also rotate the mandible to the right and to the left while at the same time keeping a downward force. This helps to center the discs in place. It usually requires more than one manipulation in order to establish correct relationships. Measurement of range of motion after every manipulation ensures that normal range has been achieved.

Where anesthesia is not indicated, or is contra-indicated, the joint may be numbed by injecting 2 CC's of Lidocaine in dental formation without epinephrine into the joint space. This can be accomplished by, in a close locked situation with the patient's mouth opened as much as possible, place the index finger into the TM posterior joint space at its maximum opening and hold that position. Clean this area with alcohol or iodine. Place a wedge in the patient's mouth to stabilize the position. Spray Fluromethane on the area beneath the index finger. A 30 gauge needle is inserted at a 45 degree angle to the tissue (laterally) and a 45 degree angle superiorly to engage the superior joint compartment. Slowly express 2 CC's into the compartment. This will provide hydraulic pressure to assist in the recapturing process. It also helps to numb the nociceptors, which would otherwise trigger the elevator muscles to inhibit the manipulation process.

Throughout treatment pain is managed 15 by means of control of inflammation and parafunctional activity. Use of pharmacology is limited to the lowest dose necessary to achieve symptom relief 16. Phonetic Bite registration is used in combination with Pulsed Radio Frequency Energy 14 to reduce inflammation and pain of injured joints

In addition to disc recapture 9, the primary method to correct skeletal, muscular, tendon and ligament asymmetries is Orthotic therapy 10.

Orthotic therapy 10 is performed by prescribing orthopedic appliances used to support, align, prevent, or correct deformities, or to improve the function of the joint, produced through a tri-planner analysis wherein the maxilla, plane of occlusion, mandible, and glenoid fossa are evaluated in order to restore the teeth in the mandibular arch to meet the corrections in the maxilla.

The purpose of Orthotic therapy is to create proper spacing between condyle and fossa where a deficiency has produced inflammatory changes. The condyle, compressing the vascular bed, reduces the blood flow available for diffusion into the synovial articular surfaces. An appliance is proper if it relieves this compression and allows blood to flow so that these injured tissues may regenerate.

Appliances may be used as a means of disc recapture, to hold the position after disc recapture, or to realign the mandible, all with the primary goal of creating proper spacing between condyle and fossa.

In addition to low dose pharmacology, the present invention embodies methods of pain management including iontophoresis, phonophoresis, trigger point and prolotherapy injections, ganglion injections, ultrasound, infra-red, acupuncture, spray and stretch, and diagnostic injections 13.

For those patients who continue to suffer from inflammation after all of the foregoing methods have been employed, the present invention embodies the method of arthrocentesis 17. Arthrocentesis is the lavage of the inflamed joint using ‘Ringers’ solution. This, in combination with Orthotic therapy, has proven to relieve patients of the most chronic and painful TMJ.

The technique is quite simple and best done under IV conscious sedation or general sedation. An antibiotic (Keflex) is introduced parentally prior to the procedure. After sedation but prior to the procedure, the superior joint compartment is inflated using a 30 gauge needle and 2% Lidocaine. Inflating the superior compartment makes it easier for cannula placement. A needle and syringe with ringers solution is inserted into the superior joint compartment (18 to 20 gauge) from a posterior approach. A second needle (cannula) is inserted into the superior joint compartment from anterior approach. This solution is used to irrigate and lavage the compartment and pass out the cannula. This procedure washes away the caustic inflammatory components that are preventing healing. This procedure is necessary because it is difficult for the body to drain these inflammatory products as the temporomandibular joint is a closed capsule. The superior joint compartment, which is responsible for translation is now cleaned, rehydrated, and is now ready for a steroid wash of Triamcinolone (Kenalog).

If the patient had a problem with limited opening, manual manipulation under anesthesia can now be accomplished. This procedure is far more efficacious with the patient unconscious and muscles relaxed. This allows us to determine whether the limited opening is due to muscle splinting or fibrous adhesions.

This technique is superior to a steroid joint injection due to the concentration of steroid in the capsule. Steroids are quite caustic and with the poor drainage in the TMJ necrosis of the condyle is likely. In fact, it is standard of care to not inject a joint with steroids more than twice, for this reason.

This procedure is best done by an oral surgeon who is comfortable with these techniques. The amount of time to accomplish these procedures bilaterally is usually 40 minutes to one hour. Due to the sedation, the patient will have to be driven home. The patient is placed on anti-inflammatories and prescriptions for analgesics may be given.

It is imperative that this procedure is followed by vigorous physical therapy to maintain ranges of motion and prevent adhesions.

Post rehabilitative appliances are essential for this process. These appliances must be tried in and techniques for range of motion exercises given to the patient prior to arthrocentesis. The patient must be instructed to hold maximum opening using the arms of the device for 30 seconds. There is a 30 second rest between repetitions. The patient should be instructed to do four sets of maximum opening in succession. Then four sets of right lateral movement followed by four sets of left lateral movement. These exercises should be done every hour post arthrocentesis. The patient should be reevaluated by the health practitioner responsible for their ongoing therapy (not oral surgeon) the next day or as soon as possible.

### CONCLUSIONS

The present invention eliminates the need for repetitive procedures and other surgeries.

What is claimed is:

1. A method and system of diagnosis of temporomandibular joint dysfunction and orofacial pain, comprising the steps of:
(a) obtaining a complete patient medical history including a description of chief complaints, prior treatment, medication usage, past trauma; and
(b) conducting a comprehensive physical examination to locate the source of pain 4 and identify any dysfunction in the masticatory system including a general examination of the face, head, and neck, cranial nerve screening, autonomic nervous system evaluation, intra-oral evaluation, range of motion, Joint Vibration Analysis, posture evaluation, recording of vitals, and a complete series of radiographic imaging;
wherein no treatment is undertaken without a complete diagnosis, which includes an understanding of how dysfunction of the TM joint can impact the entire body, and determination that any injury to the TM joint is primary.

2. The method of claim 1 wherein said step (a) includes a complete headache and pain history including location, severity, and frequency.

3. The method of claim 1 wherein said step (a) includes a determination of whether symptoms are chronic or acute, diurnal or nocturnal.

4. The method of claim 1 wherein said step (b) includes the measuring of tonus of muscles by using electromyography.

5. The method of claim 1 wherein said step (b) includes the measuring of mandibular movement by using electrognathology.

6. The method of claim 1 wherein said step (b) includes autonomic testing to triage the injuries and prioritize treatment.

7. The method of claim 1 wherein said step (b) includes the phonetic ‘S’ position of speech to determine proper condyle fossa relationship and to improve passage of airway.

8. A method and system of treatment of temporomandibular joint dysfunction and orofacial pain, comprising the steps of:
(a) Recapturing discs using principles of gentle body mechanics;
(b) Re-positioning the mandible through orthotic and physical therapy to restore function and reduce symptoms; and
(c) managing pain by means of control of inflammation and parafunctional activity.

9. The method of claim 8, wherein said step (a) is performed by manual manipulation consisting of depressing the mandible and simultaneously rotating the mandible to the right and the left while maintaining a downward force.

10. The method of claim 8, wherein said step (b) is performed by prescribing orthotics produced through a tri-planner analysis wherein the maxilla, plane of occlusion, mandible, and glenoid fossa are evaluated in order to restore the teeth in A, the mandibular arch to meet the corrections in the maxilla.

11. The method of claim 8, wherein said step (c) is performed by limiting use of pharmacology to the lowest dose necessary to achieve symptom relief and focusing treatment on relief of inflammation, support of parafunctional activity and mechanical prevention of trauma. Phonetic Bite registration is used in combination with Pulsed Radio Frequency Energy to reduce inflammation and pain of injured joints.
